SEQUENCE LISTING (28 sequences)

SEQ ID NO: 1 - protein, 584 aa, Artificial Sequence
  synthetic sequence
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLGGGGSGGGGSGGGGSCEKPYACPECGKSFSQSSHLVRHQRTHTGEKPYKCPECGKSFSDCRDLARHQRTHTGEKPYKCPECGKSFSRSDKLVRHQRTHTGKKMEKPYACPECGKSFSRKDSLVRHQRTHTGEKPYKCPECGKSFSQSGDLRRHQRTHTGEKPYKCPECGKSFSDCRDLARHQRTHTGESGGGGSGGGGLKYNLTEFKKTKSNINKASLIGYSCFESYGYVYDPEFTGPTLTASGANSRIKIKDGSNIRKMNSDETFLYMGFDSQDGKRVNEIEFLTENQKIFVCGNSISVEVLEAIIDKIGG

SEQ ID NO: 2 - protein, 584 aa, Artificial Sequence
  synthetic sequence
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLGGGGSGGGGSGGGGSCEKPYACPECGKSFSQSSHLVRHQRTHTGEKPYKCPECGKSFSDCRDLARHQRTHTGEKPYKCPECGKSFSRSDKLVRHQRTHTGKKMEKPYACPECGKSFSRKDSLVRHQRTHTGEKPYKCPECGKSFSQSGDLRRHQRTHTGEKPYKCPECGKSFSDCRDLARHQRTHTGESGGGGSGGGGLKYNLTEFKKTKSNINKASLIGYSPFCSYGYVYDPEFTGPTLTASGANSRIKIKDGSNIRKMNSDETFLYMGFDSQDGKRVNEIEFLTENQKIFVCGNSISVEVLEAIIDKIGG

SEQ ID NO: 3 - DNA, 1754 bp, Artificial Sequence
  synthetic sequence
atgagcaaagtagaaaataaaacaaaaaaacttagagtatttgaagcttttgctggaattggtgctcaaagaaaagccttggagaaagtcagaaaagatgaatatgaaatagtagggcttgctgaatggtatgttcctgcaattgttatgtatcaagctatacacaacaattttcatacaaagttggagtataaatcagtttctagagaagaaatgattgactatttggaaaataaaacactatcttggaactcaaaaaatccagtatctaatggttattggaagagaaaaaaagatgatgaacttaaaattatatataatgcaattaagttatctgaaaaagagggtaatatttttgatattagagacctttacaaaagaactttgaaaaatatagatttattaacatattcatttccttgtcaagacttatctcaacagggtattcaaaagggtatgaaaagaggttctggtactagatcaggtctcttatgggaaattgaaagagctttggattcaactgaaaaaaatgacttaccaaaatacttgttaatggaaaatgtaggggctcttcttcacaagaagaatgaagaagaactaaatcaatggaagcaaaaattagaaagtcttggctatcaaaactcaattgaagttttgaatgccgctgacttcggttcctcacaagcaagaagaagagtttttatgatatctactttaaatgaatttgttgaactaccaaagggagataaaaaacctaaaagtatcaaaaaagttttaaataaaatagtttctgaaaaagatattttaaataatttaggcggtggaggatccggaggcggtggtagcggtggaggaggctcttgcgagaaaccgtacgcatgtccggagtgcggtaagagcttcagccagtccagccacctggtccgccaccagcgtacccacactggtgaaaaaccatataaatgccctgaatgtggtaaaagcttctctgattgccgcgacctggcacgtcatcagcgcacccataccggcgaaaaaccgtacaaatgcccggaatgcggtaaatctttcagccgttccgacaaactggtacgccatcaacgtactcatactggtaaaaaatggaaaaaccatacgcttgcccggagtgtggcaaaagctttagccgtaaggatagcctggtacgccatcagcgtacgcacactggcgaaaaaccttacaagtgcccggaatgtggcaagtctttttctcaatctggtgatctgcgtcgtcatcagcgcactcacactggtgaaaaaccgtacaaatgcccggagtgcggcaaatctttctctgattgtcgtgacctggcgcgtcaccagcgtacccacaccggtgaaagcggaggcggtggttccggcggtggaggattgaaatataatttaactgaatttaaaaaaacaaaatcaaatataaataaagcttcactgattggttacagttgttttgagtcttatggttatgtttatgatcctgaatttacaggaccaaccttaactgcaagcggtgcaaattcaagaataaaaatcaaagatggatctaatattagaaaaatgaactcagacgaaactttcttatatatggggtttgattcacaagatggaaaaagagtaaatgaaattgaatttttaactgaaaatcaaaaaatatttgtttgtggaaattcaatatcagtagaagttttggaagcgattatagataaaattggaggttaa

SEQ ID NO: 4 - DNA, 1755 bp, Artificial Sequence
  synthetic sequence
atgagcaaagtagaaaataaaacaaaaaaacttagagtatttgaagcttttgctggaattggtgctcaaagaaaagccttggagaaagtcagaaaagatgaatatgaaatagtagggcttgctgaatggtatgttcctgcaattgttatgtatcaagctatacacaacaattttcatacaaagttggagtataaatcagtttctagagaagaaatgattgactatttggaaaataaaacactatcttggaactcaaaaaatccagtatctaatggttattggaagagaaaaaaagatgatgaacttaaaattatatataatgcaattaagttatctgaaaaagagggtaatatttttgatattagagacctttacaaaagaactttgaaaaatatagatttattaacatattcatttccttgtcaagacttatctcaacagggtattcaaaagggtatgaaaagaggttctggtactagatcaggtctcttatgggaaattgaaagagctttggattcaactgaaaaaaatgacttaccaaaatacttgttaatggaaaatgtaggggctcttcttcacaagaagaatgaagaagaactaaatcaatggaagcaaaaattagaaagtcttggctatcaaaactcaattgaagttttgaatgccgctgacttcggttcctcacaagcaagaagaagagtttttatgatatctactttaaatgaatttgttgaactaccaaagggagataaaaaacctaaaagtatcaaaaaagttttaaataaaatagtttctgaaaaagatattttaaataatttaggcggtggaggatccggaggcggtggtagcggtggaggaggctcttgcgagaaaccgtacgcatgtccggagtgcggtaagagcttcagccagtccagccacctggtccgccaccagcgtacccacactggtgaaaaaccatataaatgccctgaatgtggtaaaagcttctctgattgccgcgacctggcacgtcatcagcgcacccataccggcgaaaaaccgtacaaatgcccggaatgcggtaaatctttcagccgttccgacaaactggtacgccatcaacgtactcatactggtaaaaagatggaaaaaccatacgcttgcccggagtgtggcaaaagctttagccgtaaggatagcctggtacgccatcagcgtacgcacactggcgaaaaaccttacaagtgcccggaatgtggcaagtctttttctcaatctggtgatctgcgtcgtcatcagcgcactcacactggtgaaaaaccgtacaaatgcccggagtgcggcaaatctttctctgattgtcgtgacctggcgcgtcaccagcgtacccacaccggtgaaagcggaggcggtggttccggcggtggaggattgaaatataatttaactgaatttaaaaaaacaaaatcaaatataaataaagcttcactgattggttacagtcctttttgttcttatggttatgtttatgatcctgaatttacaggaccaaccttaactgcaagcggtgcaaattcaagaataaaaatcaaagatggatctaatattagaaaaatgaactcagacgaaactttcttatatatggggtttgattcacaagatggaaaaagagtaaatgaaattgaatttttaactgaaaatcaaaaaatatttgtttgtggaaattcaatatcagtagaagttttggaagcgattatagataaaattggaggttaa

SEQ ID NO: 5 - DNA, 6794 bp, Artificial Sequence
  synthetic sequence
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcttaatcatatacgtatattagtttcgcagatctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcctccggctcgtatgttgtgtggaattgtgagcggataacaatttcacacaggaaacagctatgaccatgattacgccaagctcgaaattaaccctcactaaagggaacaaaagctggagctccaccgcggtggcggccgcatagatttcaaggagacagtccatatgagcaaagtagaaaataaaacaaaaaaacttagagtatttgaagcttttgctggaattggtgctcaaagaaaagccttggagaaagtcagaaaagatgaatatgaaatagtagggcttgctgaatggtatgttcctgcaattgttatgtatcaagctatacacaacaattttcatacaaagttggagtataaatcagtttctagagaagaaatgattgactatttggaaaataaaacactatcttggaactcaaaaaatccagtatctaatggttattggaagagaaaaaaagatgatgaacttaaaattatatataatgcaattaagttatctgaaaaagagggtaatatttttgatattagagacctttacaaaagaactttgaaaaatatagatttattaacatattcatttccttgtcaagacttatctcaacagggtattcaaaagggtatgaaaagaggttctggtactagatcaggtctcttatgggaaattgaaagagctttggattcaactgaaaaaaatgacttaccaaaatacttgttaatggaaaatgtaggggctcttcttcacaagaagaatgaagaagaactaaatcaatggaagcaaaaattagaaagtcttggctatcaaaactcaattgaagttttgaatgccgctgacttcggttcctcacaagcaagaagaagagtttttatgatatctactttaaatgaatttgttgaactaccaaagggagataaaaaacctaaaagtatcaaaaaagttttaaataaaatagtttctgaaaaagatattttaaataatttaggcggtggaggatccggaggcggtggtagcggtggaggaggctcttgcgagaaaccgtacgcatgtccggagtgcggtaagagcttcagccagtccagccacctggtccgccaccagcgtacccacactggtgaaaaaccatataaatgccctgaatgtggtaaaagcttctctgattgccgcgacctggcacgtcatcagcgcacccataccggcgaaaaaccgtacaaatgcccggaatgcggtaaatctttcagccgttccgacaaactggtacgccatcaacgtactcatactggtaaaaagtaaccatggaaatgcataagtgaataaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgagacgatgatcggcctgtcgcttgcggtattcggaatcttgcacgccctcgctcaagccttcgtcactggtcccgccaccaaacgtttcggcgagaagcaggccattatcgccggcatggcggccgacgcgatgggctacgtcttgctggcgttcgcgacgcgaggctggatggccttccccattatgattcttctcgcttccggcggcatcgggatgcccgcgtttcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggtgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaagggcccgggccactgcggctgcgcactccggccccgctgaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaatagttacggcttatgacatctttgtggacacatcattcactttttattcacatccggccctgaactcgctaggacttgccccggtgcattttttaaatacccgcgaaaaatagagctgatcgtcaaatccaacattgcgcccaacggtcgctatcggcattcgcgtagtgctaagcagaagtttcgcctggctgatacgctgatcttcgcgccagctcaatacgctaatgcctaactgctggcggaacagatgtgataaccgggagggcgacaggcagacatgctgggcgacgctggcgatatcaaaatggctgtccgccagatggtcgctgatatactggcaggcatcgcgcacacggctatccatcggcgggtgcaacgactcattaattaccgccatacgtctgagcaacaactgctccagcagattgatcgccagtagctcagaatagcgaccttccccttgcccggcgctgatgatctgcccgaacagttcgctgaaatgcggctggcgcgcctcgtccgggcggaaaaatcctgtctgggcaaagattgtcggccaggtcagccactcctgccagtaggcgcgaggccggaaataaacccactggtgataccactcgctggcgtccggatgccgtccatagtgatgaatctcgcccggcggaaacaataatatatcgccaggccgacagacaaactgctcgccattattattaatgacgccctctccgcggatggtcaggttaagaatatatcccttcatgcccaacggacgatcgataaaaaaatccagatatccattcgcttcgatcggcgtcagcccggcgaccagatgggcattaaatgaatatcccggcaatagcggatcattttgcgtttcagccatgatttctctaccccccgatgttcagagaagaaacaaattgtccatatcgaccaggacgacagagcttccgtctccgcaagactttgcgcttgatgaaagcacgtatcaaccccgcttgtgaaaagcgctttgtaacaaaagcgtacagttcaggcgataaaattaagtaacagaagtgtctataactatggctggaatgtccacattgaatatttgcacagcgtcacactttgcaaagcattagcatttttgtccataagattagcggatcctgcctgacggtttttgccgcgactctctataatttctccatacctgtttttctggatggagtaagcatatggaaaaaccatacgcttgcccggagtgtggcaaaagctttagccgtaaggatagcctggtacgccatcagcgtacgcacactggcgaaaaaccttacaagtgcccggaatgtggcaagtctttttctcaatctggtgatctgcgtcgtcatcagcgcactcacactggtgaaaaaccgtacaaatgcccggagtgcggcaaatctttctctgattgtcgtgacctggcgcgtcaccagcgtacccacaccggtgaaagcggaggcggtggttccggcggtggaggattgaaatataatttaactgaatttaaaaaaacaaaatcaaatataaataaagcttcactgattggttacagttgttttgagtcttatggttatgtttatgatcctgaatttacaggaccaaccttaactgcaagcggtgcaaattcaagaataaaaatcaaagatggatctaatattagaaaaatgaactcagacgaaactttcttatatatggggtttgattcacaagatggaaaaagagtaaatgaaattgaatttttaactgaaaatcaaaaaatatttgtttgtggaaattcaatatcagtagaagttttggaagcgattatagataaaattggaggttaaactagtcactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcag

SEQ ID NO: 6 - DNA, 6794 bp, Artificial Sequence
  synthetic sequence
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcttaatcatatacgtatattagtttcgcagatctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcctccggctcgtatgttgtgtggaattgtgagcggataacaatttcacacaggaaacagctatgaccatgattacgccaagctcgaaattaaccctcactaaagggaacaaaagctggagctccaccgcggtggcggccgcatagatttcaaggagacagtccatatgagcaaagtagaaaataaaacaaaaaaacttagagtatttgaagcttttgctggaattggtgctcaaagaaaagccttggagaaagtcagaaaagatgaatatgaaatagtagggcttgctgaatggtatgttcctgcaattgttatgtatcaagctatacacaacaattttcatacaaagttggagtataaatcagtttctagagaagaaatgattgactatttggaaaataaaacactatcttggaactcaaaaaatccagtatctaatggttattggaagagaaaaaaagatgatgaacttaaaattatatataatgcaattaagttatctgaaaaagagggtaatatttttgatattagagacctttacaaaagaactttgaaaaatatagatttattaacatattcatttccttgtcaagacttatctcaacagggtattcaaaagggtatgaaaagaggttctggtactagatcaggtctcttatgggaaattgaaagagctttggattcaactgaaaaaaatgacttaccaaaatacttgttaatggaaaatgtaggggctcttcttcacaagaagaatgaagaagaactaaatcaatggaagcaaaaattagaaagtcttggctatcaaaactcaattgaagttttgaatgccgctgacttcggttcctcacaagcaagaagaagagtttttatgatatctactttaaatgaatttgttgaactaccaaagggagataaaaaacctaaaagtatcaaaaaagttttaaataaaatagtttctgaaaaagatattttaaataatttaggcggtggaggatccggaggcggtggtagcggtggaggaggctcttgcgagaaaccgtacgcatgtccggagtgcggtaagagcttcagccagtccagccacctggtccgccaccagcgtacccacactggtgaaaaaccatataaatgccctgaatgtggtaaaagcttctctgattgccgcgacctggcacgtcatcagcgcacccataccggcgaaaaaccgtacaaatgcccggaatgcggtaaatctttcagccgttccgacaaactggtacgccatcaacgtactcatactggtaaaaagtaaccatggaaatgcataagtgaataaggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgagacgatgatcggcctgtcgcttgcggtattcggaatcttgcacgccctcgctcaagccttcgtcactggtcccgccaccaaacgtttcggcgagaagcaggccattatcgccggcatggcggccgacgcgatgggctacgtcttgctggcgttcgcgacgcgaggctggatggccttccccattatgattcttctcgcttccggcggcatcgggatgcccgcgtttcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggtgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaagggcccgggccactgcggctgcgcactccggccccgctgaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaatagttacggcttatgacatctttgtggacacatcattcactttttattcacatccggccctgaactcgctaggacttgccccggtgcattttttaaatacccgcgaaaaatagagctgatcgtcaaatccaacattgcgcccaacggtcgctatcggcattcgcgtagtgctaagcagaagtttcgcctggctgatacgctgatcttcgcgccagctcaatacgctaatgcctaactgctggcggaacagatgtgataaccgggagggcgacaggcagacatgctgggcgacgctggcgatatcaaaatggctgtccgccagatggtcgctgatatactggcaggcatcgcgcacacggctatccatcggcgggtgcaacgactcattaattaccgccatacgtctgagcaacaactgctccagcagattgatcgccagtagctcagaatagcgaccttccccttgcccggcgctgatgatctgcccgaacagttcgctgaaatgcggctggcgcgcctcgtccgggcggaaaaatcctgtctgggcaaagattgtcggccaggtcagccactcctgccagtaggcgcgaggccggaaataaacccactggtgataccactcgctggcgtccggatgccgtccatagtgatgaatctcgcccggcggaaacaataatatatcgccaggccgacagacaaactgctcgccattattattaatgacgccctctccgcggatggtcaggttaagaatatatcccttcatgcccaacggacgatcgataaaaaaatccagatatccattcgcttcgatcggcgtcagcccggcgaccagatgggcattaaatgaatatcccggcaatagcggatcattttgcgtttcagccatgatttctctaccccccgatgttcagagaagaaacaaattgtccatatcgaccaggacgacagagcttccgtctccgcaagactttgcgcttgatgaaagcacgtatcaaccccgcttgtgaaaagcgctttgtaacaaaagcgtacagttcaggcgataaaattaagtaacagaagtgtctataactatggctggaatgtccacattgaatatttgcacagcgtcacactttgcaaagcattagcatttttgtccataagattagcggatcctgcctgacggtttttgccgcgactctctataatttctccatacctgtttttctggatggagtaagcatatggaaaaaccatacgcttgcccggagtgtggcaaaagctttagccgtaaggatagcctggtacgccatcagcgtacgcacactggcgaaaaaccttacaagtgcccggaatgtggcaagtctttttctcaatctggtgatctgcgtcgtcatcagcgcactcacactggtgaaaaaccgtacaaatgcccggagtgcggcaaatctttctctgattgtcgtgacctggcgcgtcaccagcgtacccacaccggtgaaagcggaggcggtggttccggcggtggaggattgaaatataatttaactgaatttaaaaaaacaaaatcaaatataaataaagcttcactgattggttacagtcctttttgttcttatggttatgtttatgatcctgaatttacaggaccaaccttaactgcaagcggtgcaaattcaagaataaaaatcaaagatggatctaatattagaaaaatgaactcagacgaaactttcttatatatggggtttgattcacaagatggaaaaagagtaaatgaaattgaatttttaactgaaaatcaaaaaatatttgtttgtggaaattcaatatcagtagaagttttggaagcgattatagataaaattggaggttaaactagtcactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcag

SEQ ID NO: 7 - protein, 5 aa, Artificial Sequence
  synthetic sequence
KFNSE

SEQ ID NO: 8 - DNA, 513 bp, Artificial Sequence
  synthetic sequence
tgcgagaaaccgtacaaatgtccggagtgcggtaagagcttcagcgattgccgtgatctggcgcgccaccagcgtacccacactggtgaaaaaccatataaatgccctgaatgtggtaaaagcttctctcgttctgatgacctggtccgtcatcagcgcacccataccggcgaaaaaccgtacaaatgcccggaatgcggtaaatctttcagccagtccagcaacctggttcgccatcaacgtactcatactggcgagaaaccgtacaaatgtccggagtgcggtaagagcttcagcacctctggcgaactggtccgccaccagcgtacccacactggtgaaaaaccatataaatgccctgaatgtggtaaaagcttctctcagcgtgcgcacctggaacgtcatcagcgtacccataccggcgaaaaaccgtacaaatgcccggaatgcggtaaatctttcagccaggcgggccatctggcgagccatcaacgtactcatactggtaaaaag

SEQ ID NO: 9 - DNA, 261 bp, Artificial Sequence
  synthetic sequence
atggaaaaaccatacaaatgcccggagtgtggcaaaagctttagccaggcgggtcatctggcgagccatcagcgtacgcacactggcgaaaaaccttacaagtgcccggaatgtggcaagtctttttctcagcgtgcacatctggaacgtcatcagcgcactcacactggtgaaaaaccgtacaaatgcccggagtgcggcaaatctttctctcagcgtgcacatctggaacgtcaccagcgtacccacaccggtgaatcc

SEQ ID NO: 10 - protein, 15 aa, Artificial Sequence
  synthetic sequence
GGGGSGGGGSGGGGS

SEQ ID NO: 11 - protein, 10 aa, Artificial Sequence
  synthetic sequence
SGGGGSGGGG

SEQ ID NO: 12 - DNA, 816 bp, Artificial Sequence
  synthetic sequence
atgagcaaagtagaaaataaaacaaaaaaacttagagtatttgaagcttttgctggaattggtgctcaaagaaaagccttggagaaagtcagaaaagatgaatatgaaatagtagggcttgctgaatggtatgttcctgcaattgttatgtatcaagctatacacaacaattttcatacaaagttggagtataaatcagtttctagagaagaaatgattgactatttggaaaataaaacactatcttggaactcaaaaaatccagtatctaatggttattggaagagaaaaaaagatgatgaacttaaaattatatataatgcaattaagttatctgaaaaagagggtaatatttttgatattagagacctttacaaaagaactttgaaaaatatagatttattaacatattcatttccttgtcaagacttatctcaacagggtattcaaaagggtatgaaaagaggttctggtactagatcaggtctcttatgggaaattgaaagagctttggattcaactgaaaaaaatgacttaccaaaatacttgttaatggaaaatgtaggggctcttcttcacaagaagaatgaagaagaactaaatcaatggaagcaaaaattagaaagtcttggctatcaaaactcaattgaagttttgaatgccgctgacttcggttcctcacaagcaagaagaagagtttttatgatatctactttaaatgaatttgttgaactaccaaagggagataaaaaacctaaaagtatcaaaaaagttttaaataaaatagtttctgaaaaagatattttaaataattta

SEQ ID NO: 13 - DNA, 345 bp, Artificial Sequence
  synthetic sequence
ttgaaatataatttaactgaatttaaaaaaacaaaatcaaatataaataaagcttcactgattggttacagtaaatttaattcagaaggttatgtttatgatcctgaatttacaggaccaaccttaactgcaagcggtgcaaattcaagaataaaaatcaaagatggatctaatattagaaaaatgaactcagacgaaactttcttatatatggggtttgattcacaagatggaaaaagagtaaatgaaattgaatttttaactgaaaatcaaaaaatatttgtttgtggaaattcaatatcagtagaagttttggaagcgattatagataaaattggaggttaa

SEQ ID NO: 14 - DNA, 37 bp, Artificial Sequence
  synthetic sequence
tagtgagcggccgctaagttggagagggaggatttga

SEQ ID NO: 15 - DNA, 46 bp, Artificial Sequence
  synthetic sequence
tagtttgaattccataaacaactacctaaacatacataacctaacc

SEQ ID NO: 16 - DNA, 45 bp, Artificial Sequence
  synthetic sequence
tgagtgcggccgcataaaataaacacaataacaatctccactctc

SEQ ID NO: 17 - DNA, 44 bp, Artificial Sequence
  synthetic sequence
ttgtatgaattcaggttgtaattttgagtagtagaggagtttag

SEQ ID NO: 18 - protein, 5 aa, Artificial Sequence
  synthetic sequence
PFCSY

SEQ ID NO: 19 - protein, 5 aa, Artificial Sequence
  syntheric sequence
CFESY

SEQ ID NO: 20 - protein, 5 aa, Artificial Sequence
  synthetic sequence
SYSSS

SEQ ID NO: 21 - protein, 87 aa, Artificial Sequence
  synthetic sequence
CEKPYACPECGKSFSQSSHLVRHQRTHTGEKPYKCPECGKSFSDCRDLARHQRTHTGEKPYKCPECGKSFSRSDKLVRHQRTHTGKK

SEQ ID NO: 22 - protein, 86 aa, Artificial Sequence
  synthetic sequence
MEKPYACPECGKSFSRKDSLVRHQRTHTGEKPYKCPECGKSFSQSGDLRRHQRTHTGEKPYKCPECGKSFSDCRDLARHQRTHTGE

SEQ ID NO: 23 - protein, 171 aa, Artificial Sequence
  synthetic sequence
CEKPYKCPECGKSFSDCRDLARHQRTHTGEKPYKCPECGKSFSRSDDLVRHQRTHTGEKPYKCPECGKSFSQSSNLVRHQRTHTGEKPYKCPECGKSFSTSGELVRHQRTHTGEKPYKCPECGKSFSQRAHLERHQRTHTGEKPYKCPECGKSFSQAGHLASHQRTHTGKK

SEQ ID NO: 24 - protein, 87 aa, Artificial Sequence
  synthetic sequence
MEKPYKCPECGKSFSQAGHLASHQRTHTGEKPYKCPECGKSFSQRAHLERHQRTHTGEKPYKCPECGKSFSQRAHLERHQRTHTGES

SEQ ID NO: 25 - DNA, 45 bp, Artificial Sequence
  synthetic sequence
aagacagagctcaaactaaataaccttccccattataattcttct

SEQ ID NO: 26 - DNA, 56 bp, Artificial Sequence
  synthetic sequence
ccgtagccatggtatatttttaataaattttttagggaaataggttaggtttttat

SEQ ID NO: 27 - DNA, 49 bp, Artificial Sequence
  synthetic sequence
aagacagagctcctctactaatcctattaccaataactactaccaataa

SEQ ID NO: 28 - DNA, 57 bp, Artificial Sequence
  synthetic sequence
ccgtagccatgggtaaagtttggggtgtttaatgagtgagttaatttatattaattg

(19) United States
(12) Patent Application Publication
(10) Pub. No.: US 2017/0058268 A1
(43) Pub. Date: Mar. 2, 2017

(54) COMPOSITIONS FOR TARGETED DNA METHYLATION AND THEIR USE

(71) Applicant: THE JOHNS HOPKINS UNIVERSITY, Baltimore, MD (US)

(72) Inventors: Marc Ostermeier, Baltimore, MD (US); Brian Chaikind, Baltimore, MD (US)

(21) Appl. No.: 15/124,917

(22) PCT Filed: Mar. 11, 2015
(86) PCT No.: PCT/US2015/019913

§ 371 Date: Sep. 9, 2016

Related U.S. Application Data
(60) Provisional application No. 61/951,196, filed on Mar. 11, 2014.

(51) Int. Cl. C12N 9/10; C12N 15/62; C12Q 1/68; C12Q 1/48

(52) U.S. Cl. CPC C12N 9/1007; C12Q 1/48; C12N 15/62; C12Q 1/6809; G01N 2333/91011; C07K 2319/81

(57) ABSTRACT

The present invention provides an in vitro directed evolution selection system to create modified methyltransferases which improve methyltransferase specificity and use it to optimize and provide fusion proteins comprising a zinc finger methyltransferase derived from M.SssI. The resulting fusion proteins show increased target methylation specificity and greatly decreased non-target methylation compared to wild-type enzyme activity. Methods of use of such fusion proteins in both prokaryotic and eukaryotic cells are also provided.

### REFERENCE TO RELATED APPLICATIONS

This application claims the benefit of U.S. Provisional Patent Application No. 61/951,196, filed on Mar. 11, 2014, which is hereby incorporated by reference for all purposes as if fully set forth herein.

### STATEMENT OF GOVERNMENTAL INTEREST

This invention was made with government support under grant no. R01GM066972 awarded by the NIH. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Mar. 5, 2014, is named P12866-01_ST25.txt and is 43,145 bytes in size.

### BACKGROUND OF THE INVENTION

CpG methylation is one of the most extensively studied epigenetic modifications and broadly regulates or maintains transcriptional activity. It is involved in proper cellular differentiation, heterochromatin formation and chromosomal stability. Further, aberrant methylation patterns cause or are observed in numerous diseases. Imprinting defects lead to disorders such as Prader-Willi and Angelman syndromes. Notably, global genomic hypomethylation and local hypermethylation of CpG islands (CGIs) commonly occur in cancer. Though much has been learned about how methylation patterns are established and erased, the causes of aberrant methylation and the reestablishment of methylation patterns during development remain active areas of research. To study the effects and dynamics of DNA methylation, it would be generally useful to target methylation toward specific, user-defined sequences.

Several groups have engineered methyltransferases that bias methylation towards user-defined DNA sequences. The general strategy, pioneered by Xu and Bestor, involves fusion of a sequence specific DNA binding domain to a methyltransferase enzyme (Nat. Genet., 17: 376-378 (1997)). These constructs have been used to affect methylation, in vitro, in E. coli, and in cancer cell lines. Biased methyltransferases have been shown to stably and heritably reduce the expression of Sox2 and Maspin genes. Siddique et al. demonstrated that targeting methylation towards the VEGF-A promoter significantly reduced gene expression in SOKV3 cells (J. Mol. Biol., 425: 479-491 (2013)). A recent review summarizes much of the literature on targeted methylation (Nucleic Acids Res., 40: 10596-10613 (2012)). Most engineered methyltransferases methylate multiple CpG sites adjacent to the desired target site on the DNA. Despite the successes of these studies in biasing methylation to a particular region, little work has focused on targeting methylation to single CpG sites.

In addition to studying effects on transcription, an engineered methyltransferase that specifically methylates a single site in a promoter would, at a minimum, be generally useful for studying the effects of single aberrant methylation events on the propagation, maintenance, and correction of epigenetic marks. Thus, there is still an unmet need for development of targeted methyltransferases to site-specifically label DNA.

### SUMMARY OF THE INVENTION

In accordance with an embodiment, the present inventors developed a strategy for achieving single-site, targeted methylation by assembly of a heterodimeric methyltransferase fusion protein that is dependent on specific DNA sequences flanking a site to be methylated. To accomplish this task, natural or artificially split DNA methyltransferases were used and these heterodimers were engineered to reduce their innate ability to reassemble into a functional enzyme. Reducing the ability of the fragments to self-assemble in a functional form is necessary as the present inventors and others have shown that bifurcated methyltransferases are capable of unassisted reassembly into functional enzymes. These reassembly-defective fragments of the present invention are fused to DNA binding polypeptides such as zinc fingers, whose recognition sequences flank the targeted CpG site. The zinc finger domains bind to DNA, increasing the local concentration of the fused methyltransferase fragments over a targeted CpG site. Proper orientation of the methyltransferase fragment-zinc finger fusions at the target site primes the fragments for reassembly into a functional enzyme. The orientation of the fragments at the target site is affected by the topology of the fusions and the amino acid linker lengths connecting protein domains. Optimization of these parameters, as well as the reduction of the affinity of fragments for each other and for DNA, allows for the reduction of non-specific activity and promotes enzymatic reassembly at the targeted CpG site.

In addition, the present inventors provide a selection strategy to improve the targeting of methyltransferases to new sites and use this strategy to optimize a M.SssI fusion construct. In an embodiment, a negative selection against off-target methylation and a positive selection for methylation at a target site in vitro. This inventive strategy allows quick identification of variants with improved targeting ability and activity in vivo. The present inventors also demonstrate the modularity of the fusion protein constructs of the present invention, by altering the zinc finger domains to redirect methylation toward a new target site.

Thus, In accordance with an embodiment the present invention can be used to design molecular tools to study the phenotypic effects of DNA methylation in a cell or population of cells.

In accordance with another embodiment, the present invention can be used to specifically modify DNA for in vivo and in vitro purposes.

In accordance with yet another embodiment, the present invention can be used to alter gene expression associated with disease states, and treat or mitigate those diseases.

In accordance with an embodiment, the present invention provides a fusion protein comprising: a) a polypeptide encoding an N-terminal portion of M.SssI methyltransferase; b) a polypeptide encoding a first DNA binding peptide specific for a DNA sequence of interest; c) a peptide encoding a first linker molecule which is covalently linked to the N-terminal portion of M.SssI methyltransferase and the first DNA binding peptide; d) a polypeptide encoding a C-terminal portion of M.SssI methyltransferase, wherein the C-terminal portion encodes a mutation; e) a polypeptide encoding a second DNA binding peptide specific for a DNA sequence of interest; and f) a peptide encoding a second linker molecule which is covalently linked to the C-terminal portion of the M.SssI methyltransferase and the second DNA binding peptide.

In accordance with an embodiment, the present invention provides a fusion protein comprising the amino acid sequence of SEQ ID NOS: 1 or 2.

In accordance with another embodiment, the present invention provides a nucleic acid molecule encoding the fusion protein described above.

In accordance with an embodiment, the present invention provides a nucleic acid molecule encoding the fusion protein described above comprising the nucleotide sequence of SEQ ID NOS: 3 or 4.

In accordance with a further embodiment, the present invention provides an expression vector comprising the nucleic acid molecule described above.

In accordance with an embodiment, the present invention provides an expression vector comprising the nucleotide sequence of SEQ ID NOS: 5 or 6.

In accordance with yet another embodiment, the present invention provides a micro-organism transformed with the expression vector described above.

In accordance with an embodiment, the present invention provides a method for selection of a fusion protein comprising a methyltransferase having specificity for a methylation site of interest, comprising: an E. coli cell transformed with the expression vector described above, wherein the expression vector comprises a restriction enzyme site having a target methylation site within the nucleic acid sequence of the restriction enzyme site, and wherein the restriction enzyme specific for said site can only cleave the restriction site in the absence of CpG methylation, and wherein the vector encodes DNA sequences which flank the restriction site that are specific for the DNA binding peptides encoded in the vector; expressing the polypeptides encoded by the vector in the E. coli cell; allowing the vector to become methylated by the methytransferase encoded by the vector; isolating the DNA of the vector; digesting the DNA of the vector in vitro with an endonuclease specific for said restriction site and with the endonuclease McrBC; incubating the vector DNA with the enzyme ExoIII; and isolating and purifying the remaining intact vectors.

### BRIEF DESCRIPTION OF THE DRAWINGS

FIGS. 1A-1E. Schematics of the vector, library, proteins, and selection used in these experiments. (A) The vector used in selections. The vector encodes for both heterodimeric fragments fused to zinc fingers under the control of separate inducible arabinose (pBAD) and IPTG (lac) promoters, a target site, and the araC gene. (B) A schema of the zinc finger-fused, bifurcated M.SssI and the mutagenized codons used in library construction of the present invention. Codons corresponding to residues 297-301 of M.SssI (located in the C-terminal fragment) were randomized Numbering scheme is that of the wildtype M.SssI. (C) An assembled zinc finger-fused heterodimeric M.SssI methyltransferase fusion protein assembled at the target site and (D) a corresponding control site. (E) An overview of the inventive selection system used in this experiment. The schematic illustrates the fates of plasmids encoding an inactive methyltransferase fusion protein construct (left), the desired targeting methyltransferase fusion protein construct methylating the target site (middle), and a nonspecific methyltransferase fusion protein construct methylating multiple M.SssI (i.e CpG) sites.

FIGS. 2A-2D. Methylation assay for selected variants. (A) Relative locations of the target site and non-target site on a plasmid linearized by NcoI digestion. (B) The target site is comprised of the HS1 and HS2 zinc finger recognition sites flanking an internal FspI restriction site. The targeted CpG site is nested within this FspI restriction site. (C) The non-target site lacks the HS1 and HS2 recognition sequences, but contains a SnaBI restriction site with a nested CpG site for the assessment of off-target methylation. (D) The restriction endonuclease protection assay for methylation at the target and non-target site uses digestion with NcoI and either FspI or SnaBI for assessment of target and off-target methylation, respectively. FspI and SnaBI cannot digest a methylated site. Shown are results from select inventive fusion protein construct variants as well as the ‘wildtype’ heterodimeric fusion protein (i.e. the methylase enzyme having no mutations to residues 297-301) with or without a catalytically inactivating (C141S), or a catalytically compromised (Q147L) mutation.

FIGS. 3A-3B. Sequence conservation at residues 297-301 of all catalytically active selected fusion protein variants. (A) The wild type sequence for residues 297-301 of M.SssI. (B) A sequence logo of active variants.

FIGS. 4A-4D. Substitution of new zinc fingers in the fusion protein construct of the present invention targets methylation towards a new site. (A) A schematic of the designed methyltransferase is shown assembled over the new, targeted CpG site. New cognate zinc finger recognition sequences flank a CpG site nested within an FspI site. Zinc fingers CD54-31Opt and CD54a have replaced the HS 1 and HS2 zinc fingers. (B) The non-target site contains the HS1 and HS2 zinc finger recognition sites flanking a CpG site nested within a FspI restriction site (i.e. this was the target site in experiments in FIG. 2). (C) The relative locations of the target site and non-target site are shown on a plasmid linearized by NcoI digestion. (D) The restriction endonuclease protection assay for methylation at the target and non-target site for the ‘wildtype’ heterodimeric enzyme (KFNSE (SEQ ID NO: 7)) and two selected variants with mutations in the region 297-301.

FIG. 5 is a table showing a small subset of the selected amino acid variants with mutations in the region 297-301.

FIGS. 6A-6D depict the constructs for eukaryotic expression vectors. A) The pBUD mammalian expression vector with relevant gene sequences, promoters, resistance marker, and origin of replication. B) A graphical representation of the zinc finger-fused methyltransferase fragments. Flag-tags and NLS-SV40 sequences are attached to each zinc finger. Below the C-terminal fragment, an enlarged area illustrates changes made to amino acid residues 295-303. The ‘wild-type’ heterodimeric methyltransferase, a generic library variant, or a construct designed to enable golden gate cloning of optimized constructs are shown. Note that the amino acid numbering corresponds to the monomeric wild-type M.SssI construct. C) A schematic of a zinc finger-fused heterodimeric methyltransferase binding to its' target site. D) The target site for N-terminal and C-terminal heterodimeric methyltransferase fragments fused to CD54-31opt (SEQ ID NO: 8) and CD54a (SEQ ID NO: 9, respectively.

FIG. 7 shows restriction digest assays of the ‘wild-type’, optimized and inactive variants. Inactive variants lack the zinc finger-fused C-terminal fragment. Variants are digested with no enzyme, FspI or SnaBI. Panel 1 depicts plasmid DNA prior to transfection. In panel 2, plasmid DNA was recovered from transfected HEK293 cells. Top (nicked) and bottom (supercoiled) bands are indicative of methylation-dependent protection from endonuclease digestion. Pixels of control DNA and ladder were saturated. The image was inverted and image contrast proportionally altered to enable visualization of transfected plasmids.

FIG. 8 depicts a Western blot of transiently transfected HEK293 cells. Lane 1:Empty pBUD.CE.4.1; lane 2: pBUD expressing zinc finger-fused N-terminal and C-terminal ‘wild type’ fragments; lane 3: pBUD expressing only the zinc finger fused N-terminal fragment; lane 4 pBUD expressing Flag-tag-EGFP-Haps59 fusion; lane 5: empty; lane 6: MagicMark XP Western Protein Standard.

FIGS. 9A-9B show bisulfite analysis of optimized and ‘WT’ variants. Percent methylation of individual CpG sites at and adjacent to the (A) target site and (B) non-target site. Percentages at each CpG site were determined by bisulfite sequencing of n number of clones. CpG sites are numbered from 1-48 or 1-60 based on their order in the sequencing read and do not indicate the distance between sites. Asterisks indicate that one CpG site was removed due to poor sequencing quality in this region. Black, ‘WT’ heterodimeric enzyme (KFNSE); orange, PFCSY variant; blue, CFESY variant. Target and non-target CpG sites (i.e. the two sites assessed by restriction enzyme digestion assays) are indicated by arrows.

### DETAILED DESCRIPTION OF THE INVENTION

In accordance with an embodiment, the present inventors provide a fusion protein comprising: a) a polypeptide encoding an N-terminal portion of M.SssI methyltransferase; b) a polypeptide encoding a first DNA binding peptide specific for a DNA sequence of interest; c) a peptide encoding a first linker molecule which is covalently linked to the N-terminal portion of M.SssI methyltransferase and the first DNA binding peptide; d) a polypeptide encoding a C-terminal portion of M.SssI methyltransferase, wherein the C-terminal portion encodes a mutation; e) a polypeptide encoding a second DNA binding peptide specific for a DNA sequence of interest; and f) a peptide encoding a second linker molecule which is covalently linked to the C-terminal portion of the M.SssI methyltransferase and the second DNA binding peptide.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules.

“Differential expression” may mean qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST 2.0.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Target” as used herein can mean an oligonucleotide or portions or fragments thereof, which may be bound by one or more DNA binding proteins, such as zinc finger proteins, for example. In some embodiments, “target” can mean a specific sequence which has at least one CpG site which can be methylated by the methylase containing fusion proteins of the present invention.

The term “methylase” or “methyltransferase” as used herein, means an enzyme or functional fragment or portion thereof, which is capable of methylating one or more CpG sites on a nucleic acid molecule.

As used herein, the term “linker” includes a polypeptide which connects either the N-terminal fragment of the methyltransferase to the DNA binding protein, or a polypeptide which connects the C-terminal fragment of the methyltransferase to the DNA binding protein. In some embodiments the linkers can vary in length from about 5 to about 20 amino acids in length, preferably between about 10 to 15 amino acids in length.

In accordance with an embodiment, the linker which connects the N-terminal fragment of the methyltransferase to the DNA binding protein comprises 15 amino acids, and has the following sequence: GGGGSGGGGSGGGGS (SEQ ID NO: 10).

In accordance with another embodiment, the linker which connects the C-terminal fragment of the methyltransferase to the DNA binding protein comprises 10 amino acids, and has the following sequence: SGGGGSGGGG (SEQ ID NO: 11).

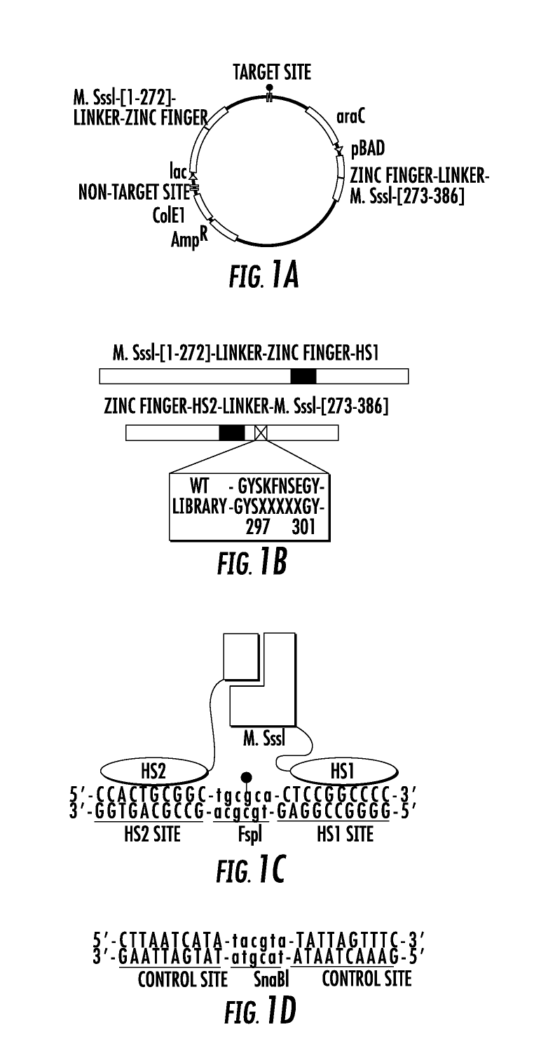

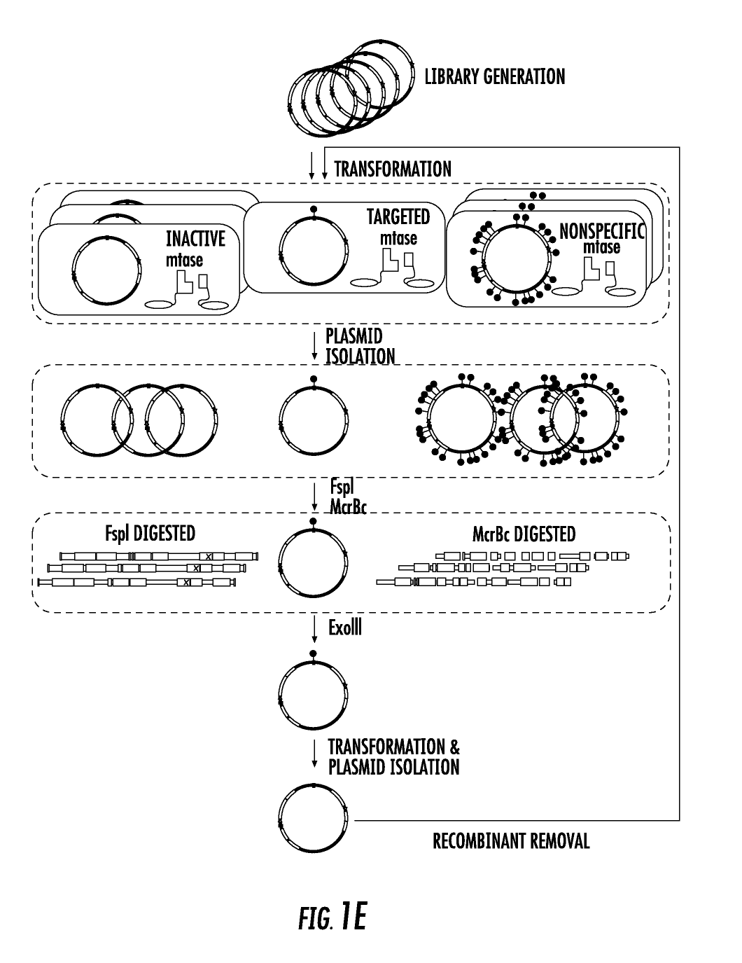

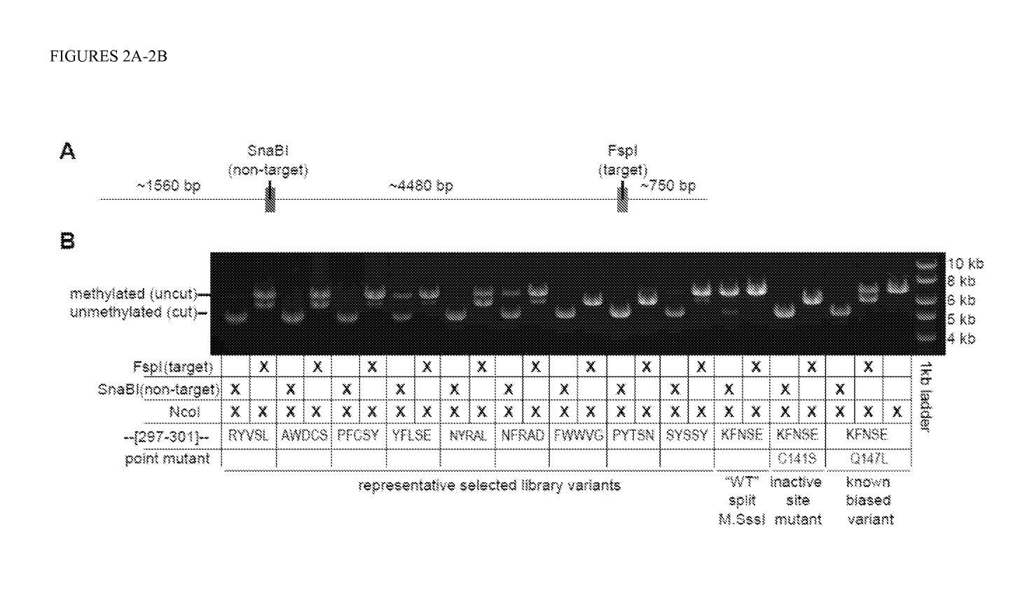

Design of the Selection System. M.SssI naturally methylates CpG sites. The inventors' previously described, bifurcated M.SssI DNA methyltransferase zinc finger fusions (FIG. 1B) biased methylation toward a targeted M.SssI site flanked by the cognate zinc finger binding sequences. However, active variants also methylated other M.SssI sites. It was sought to reduce this off-target methylation while maintaining high levels of methylation at the targeted M.SssI site. The present invention describes an in vitro selection system that preferentially enriches variants possessing the ability to methylate the target site, but lacking the ability to methylate other non-targeted M.SssI sites on the plasmid (FIG. 1D).

In vitro selection strategies have been used to enrich for methyltransferases with relaxed or altered specificity. Most strategies rely on methylation-dependent protection from restriction endonuclease digestion to positively select for DNA encoding a methyltransferase with altered specificity. The selection scheme of the present invention differs from previous studies as it additionally employs the enzyme McrBC as a negative selection against unwanted methylation activity. McrBC is a GTP-requiring, modification-dependent endonuclease of E. coll K-12, and specifically recognizes DNA sites of the form 5′ R^{m}C 3′. DNA cleavage normally requires translocation-mediated coordination between two such recognition elements at distinct sites. In our system for altering methyltransferase specificity, a single plasmid contains both genes encoding the zinc finger-fused M.SssI fragments as well as a targeted M.SssI site nested within an FspI restriction site and flanked by zinc finger binding sequences (FIGS. 1A, 1C). The plasmid also has over 400 other M.SssI (i.e. CpG) sites. Once transformed into E. coli, the methyltransferase fragments encoded by the plasmid are expressed, resulting in methylation of the same plasmid. The plasmid DNA is isolated and subjected to in vitro digestions with endonucleases FspI and McrBC (FIG. 1D). Since FspI digestion is blocked by methylation, FspI digestion serves to select for methylation at the targeted CpG site. McrBC is an endonuclease that recognizes and cleaves DNA with two distal methylated sites. McrBC will not digest a single site that is methylated or hemimethylated unless there is a second methylated site on the same DNA within about 40-3000 bp. It was therefore expected that most plasmids methylated at multiple M.SssI sites would be digested by McrBC. Thus, McrBC digestion selects against off-target methylation. The DNA is then incubated with ExoIII to degrade any plasmid that is digested at least once, ideally leaving the plasmid DNA encoding a highly specific methyltransferase intact for the subsequent transformation.

The initial proof of principal selections described herein demonstrate that McrBC, FspI and ExoIII treatment of unmethylated plasmid DNA, followed by transformation resulted in a 99.85% decrease in the number of transformants relative to untreated DNA. Similarly, McrBC, FspI and ExoIII treatment of a highly methylated plasmid reduced transformants by 99.95% relative to untreated control.

Design of the Library. A library of M.SssI C-terminal fragment variants randomized at residues 297-301 was constructed (FIG. 1B). It was hypothesized that mutations to these residues might reduce the ability of the split methyltransferase to methylate non-targeted CpG sites by reducing the fragment's inherent affinity for double-stranded DNA. Early studies indicated that M.SssI interacts with DNA, irrespective of the presence of CpG sites and subsequently methylates processively. Further, a homology model of M.SssI suggested that residues 297 and 299 form contacts with the ribose phosphate backbone on the CpG bases complementary to the methylated CpG site. Mutational studies showed that for monomeric M.SssI, K297A or N299A mutations did not appreciably affect either the catalytic activity, or the dissociation constant of a CpG containing oligonucleotide. Mutating these residues, it was thought, could eliminate the innate affinity of the fragments for DNA without affecting the catalytic activity of the enzyme.

In addition, the homology model used indicated the amide backbone of serine residue at position 300 made base-specific contacts with the cytosine and guanine bases complementary to the methylated strand. This model initially implicated serine's conserved and catalytically important role for stabilizing the complementary strand during base flipping and methylation. However, it was found that the S300P mutation resulted in only a three-fold increase in a dissociation constant and no significant change in initial rate of reaction.

### EXAMPLES

Enzymes, Oligonucleotides and Bacterial Strains. Restriction enzymes, T4 ligase,T4 kinase, and Phusion High Fidelity PCR MMX were purchased from New England Biolabs (Ipswich, Mass.). BoxI was purchased from ThermoFisher Scientific (Waltham, Mass.). Platinum Pfx DNA polymerase was purchased from Life Technologies (Carlsbad, Calif.). PfuTurbo Cx Hotstart DNA polymerase was purchase from Agilent Technologies (Santa Clara, Calif.). Plasmid-Safe-ATP-dependent DNAse was purchased from Epicentre (Madison, Wis.). pDIMN8 and pAR plasmids have been previously described (Nucleic Acids Res., 38: 1749-1759 (2010); PLoS ONE 7: e44852 (2012)). All oligonucleotides and gBlocks were synthesized by Invitrogen (Carlsbad, Calif.) or Integrated DNA Technologies (Coralville, Iowa). Gel electrophoresis and PCR were performed essentially as previously described. Plasmids were isolated using QIAprep Spin Miniprep Kit (Qiagen, Valencia, Calif.). DNA fragments were purified from agarose gels using QIAquick Gel Extraction Kit (Qiagen, Valencia, Calif.) or PureLink Quick Gel Extraction Kit (Invitrogen, Carlsbad,Calif., USA) and further concentrated using DNA Clean & Concentrator-5 (Zymo Research, Irvine, Calif.).

Escherichia coli K-12 strain ER2267 [F proA⁺B⁺lacI^{q} D(lacZ)M15 zzf::mini-Tn10 (Kan^{R})/D(argF-lacZ)U169 glnV44 c14⁻(McrA⁻) rfbD1? recA1 rclA1? cndA1 spoT148 thi-1 D(mcrC-mrr)114::IS10] was acquired from New England Biolabs (Ipswich, Mass.) and was used in selections, methylation assays and cloning. NEB 10-beta Competent E. coli (High Efficiency) [Δ(ara-leu) 7697 araD139 fhuA ΔlacX74 galK16 galE15 e14-φ80dlacZΔM15 recA1 relA1 endA1 nupG rpsL (StrR) rph spoT1 Δ(mrr-hsdRMS-mcrBC)] and NEB 5-alpha Competent E. coli (High Efficiency) [fhuA2D(argF-lacZ)U169 phoA glnV44 φ80A(lacZ)M15 gyrA96 recA1 relA1 endA1 thi-1 hsdR17] were also used for cloning and purchased from New England Biolabs (Ipswich, Mass.).

Plasmid Creation. pDIMN8, was used for library creation and testing of library variants. pDIMN9 was constructed as follows for use in golden gate cloning. Plasmid pDIMN8 was altered by silently mutating a BsaI site in the Amp^{R} gene via pFunkel mutagenesis (PLoS ONE 7: e52031 (2012)). PCR, digestion and cloning removed a BbsI restriction site to create vector pDIMN9. Golden gate cloning was used to fuse new zinc finger proteins to methyltransferase fragments. For the creation of plasmids used in golden gate cloning, regions encoding zinc finger proteins were replaced with BbsI sites. pDIMN9 contained a M.SssI[aa 1-272]-BbsI construct (SEQ ID NO: 12) for the addition of zinc fingers to the N-terminal fragment. pAR contained BbsI-M.SssI[aa 273-386] (SEQ ID NO: 13) construct for the addition of new zinc fingers to the C-terminal fragments. gBlocks encoding zinc fingers and BbsI sites were purchased from IDT. Golden gate cloning to fuse zinc finger-encoding gBlocks to the above plasmids was performed essentially as described (Nat. Protoc., 7: 171-192 (2012)). Zinc finger CD54a was designed using the zinc finger tools website and previously identified zinc finger domains. Individual C-terminal and N-terminal zinc finger-fused constructs were digested with EcoRI and Spel as previously described to place these constructs on the same plasmid for characterization in E. coli. Site 1 and site 2 were altered as previously described to vary the sequences flanking different CpG sites.

Plasmid Construction for Eukaryotic Expression. Genes encoding zinc finger-fused M.SssI heterodimeric fragments were cloned into mammalian expression vector pBUDCE4.1. The C-terminal fragment zinc finger fusion gene was placed under the control of the CMV immediate-early promoter. The N-terminal fragment zinc finger fusion gene was placed under the control of the EF-1α promoter. Oligonucleotides encoding the SV40-NLS and a FLAG-tag were annealed to their reverse complement sequence by incubating at over 95° C. for over 2 minutes and cooling to room temperature. Annealed oligonucleotides contained overhangs complementary to cut sites at either the N-termini or C-termini of the zinc fingers. Double stranded DNA was phosphorylated and ligated to fuse these DNA sequences to zinc finger genes, creating the constructs shown in FIG. 1B. The region between the origin of replication and CMV promoter was removed; we cloned various target sites in its place. These target sites were created by annealing complementary, phosphorylated oligonucleotides, as above. Oligonucleotides encoded the desired target site and, when annealed to each other, created double stranded sequences of DNA with overhangs complementary for restriction sites in the pBUD plasmid. This DNA was then ligated into pBUD plasmids. The above plasmid was modified with a Type IIS restriction enzyme, BsmBI, in order clone and test optimized variants that were identified through E. coli selections described herein. A gBlock of the CD54a-fused-Cterminal M.SssI fragment was designed; within this gBlock, two adjacent BsmBI sites separated by an internal sequence replaced the region encoding amino acids [297-301]. This gBlock was then cloned into and replaced the zinc finger-fused-C-terminal M.SssI fragment in the pBUD vector. The internal sequence between the two BsmBI sites was later also altered to remove an unwanted DNA sequence. The final construct is shown in FIG. 1B.

The above plasmid was used to construct optimized C-terminal constructs, following a golden gate procedure performed essentially as described previously. In order to insert novel DNA sequences in the region encoding wildtype residues 297-301, variant sequences were created by designing two complementary oligonucleotides, annealed as above. These oligonucleotides contained sequences encoding novel amino acids flanked by regions complementary to BsmBI cut sites in the plasmid. BsmBI sites were then placed outside of these complementary regions. Digestion of BsmBI in the presence of the plasmid, the annealed oligonucleotides and T7 ligase allowed for the rapid creation of optimized C-terminal fragments into the pBUD mammalian vectors.

Eukaryotic Cell Culture. HEK293 cells were grown in RPMI 1640 with glutamine (Cat #11875-093, Life Technologies, Carlsbad, Calif.) supplemented with 10% FBS (Hyclone Cat #SH30088.03, Thermo Scientific, Waltham, Mass.). RKO cells were obtained from the American Type Culture Collection (Manassas, Va.). Cells were grown in Minimal Essential Media with Earles (E-MEM) balanced salts and glutamine (Cat#112-018-101, Quality Biologicals, Gaithersburg, Md.) supplemented with 10% FBS. Cells were grown at 5% CO₂ and at 37° C. Cells were split by washing with DPBS (Cat #14190-250, Life Technologies, Carlsbad, Calif.), adding 1-2 mL 0.25% Trpsin-EDTA Cat #25-053-C1 (MediaTech, Herndon, Va.) and diluting in appropriate media. Cells were frozen by trypsinizing, diluting in complete media and adding 5% DMSO before storage o/n at −80° C. Cells were then transferred and stored in liquid nitrogen.

Transfection into HEK293 and RKO cells. Cells were transfected with Lipofectamine 2000 Transfection Reagent (Life Technologies, Carlsbad, Calif.). DNA used for transient transfections was isolated from E. coli cultured in low salt media at pH 7.5, and supplemented with 50 μg/ml zeocin (Life Technologies, Carlsbad, Calif.). Plasmid was isolated with the PureYield Plasmid Miniprep Sytem (Promega, Madison, Wis.) according to the large culture volume protocol. The day before transfection, HEK293 cells were seeded into 6-well plates (6×10⁵ cells/well) or 10 cm dishes (3×10⁶ cells/dish) to achieve cultures of 90-95% confluency on the day of transfection. For transfections in 6-well plates, 5 μg of DNA was incubated in 625 μl Opti-MEM media (Life Technologies, Carlsbad, Calif.) for five minutes andcombined with 12.5 μl lipofectamine in 625 μl Opti-MEM, which was then incubated for at least 20 minutes at room temperature. RPMI complete media (RPMI+10% FBS) was removed and replaced with 1250 μl Opti-MEM media. The DNA, lipofectamine/Opti-MEM solution was added to cells and incubated for 24 hours at 5% CO₂ and 37° C. This protocol was scaled up six-fold for transfections in 10 cm plates.

For transient transfections of RKO cells, 5×10⁴ cells/well were seeded into 6-well plates and grown for several days until they achieved 40-60% confluency. A mixture of 2 μg of DNA in 100 μl of E-MEM was incubated for five minutes and mixed with 6 μl of lipofectamine in 100 μl of E-MEM. DNA in E-MEM was combined with lipofectamine in E-MEM and incubated at room temperature for over 20 minutes. Fresh complete media (E-MEM+10% FBS) (0.8 μl ) was added to each well before transfection. The DNA/lipofectamine/E-MEM mixture (200 μl ) was added to each well in a dropwise fashion and incubated for 24 hours at 5% CO₂ and 37° C.

For both RKO and HEK293 cells, after a 24-hour incubation of the transfection reagent and DNA, transfection mixture was replaced with 2 ml of the appropriate complete media (per well of a 6-well plate). Media was replaced, if necessary, at 24-hour intervals and the cells were harvested 72 hours after the initial addition of the transfection reagent.

Eukaryotic plasmid digestion assays. Isolation of plasmid DNA was performed as follows. Briefly, for 6-well plates, cells were disrupted mechanically or with trypsin and washed several times in DPBS. Cells were spun at 1500×g, resuspended in residual DPBS and lysed by addition of 250 μl Hirt lysis buffer (0.6% w/v SDS and 10 mM EDTA). After lysis at room temperature for 20 minutes, 100 μl of ice cold 5M NaCl was added and the mixture was incubated at 4° C. overnight. Mixture was spun at 14,000 ×g for 15 minutes.

Phenol chloroform extraction and ethanol precipitation were performed as follows. Phenol:Chloroform extraction of the aqueous layer was performed at least twice and mixtures were back extracted with TE buffer. Aqueous layers were combined and extracted with an equal volume of chloroform. Aqueous layer was supplemented with 40 mM MgCl₂ and 2 μl pellet paint co-precipitant (EMD Millipore, Billerica, Mass.) per 500 μl of aqueous solution. Three volumes of ethanol (−20° C.) per one volume of aqueous layer was added and incubated overnight at −20° C. Solution was centrifuged at 14,000 ×g and at 0° C. for 30 minutes or more. The pellet was washed once in 70% w/v ethanol and redissolved in water. The protocol was scaled 6× and slightly modified for larger 10 cm dish transfection experiments.

Isolated DNA was purified with a Zymo Clean and Concentrator-5 columns essentially as recommended by the manufacturer. Depending on size of the transfection experiment (6-well or 10 cm dish), DNA was incubated with 5 or 15 units of Plasmid-Safe-ATP-Dependent DNAse (Epicentre, Madison, Wis.) and 5 or 15 μg of DNAse and protease free RNAse (ThermoScientific, Waltham, Mass.), supplemented with 1 mM ATP and 1× Plasmid-Safe reaction buffer. Reactions were incubated for at least 1 hour at 37° C. and heat killed at over 70° C. for at least 20 minutes. Reactions were divided into three equal aliquots and incubated with SnaBI (2.5 units) supplemented with BSA, FspI (2.5 units), or no enzyme at 37° C. for 1 hour. Digestions were analyzed on a 1.2% w/v agarose gel in TAE run at 90 volts for 40 minutes. Images were captured using a Gel Logic 112 Imaging System.

Bisulfite sequencing. RKO cells, transfected with plasmid DNA, were harvested 72 hours after transfection via trypsinization and washed in DPBS. Chromosomal DNA was isolated using a Genomic DNA Extraction PureLink kit (Life Technologies, Carlsbad, Calif.) per manufacturer's instructions. Isolated DNA was treated with bisulfite DNA reagent using and EZ DNA Methylation-Gold Kit (Zymo Research, Irvine, Calif.). PfuTurbo Cx Hotstart DNA polymerase (Agilent Technologies, Santa Clara, Calif.) was used to amplify bisulfite converted DNA. Touch down PCR was used to amplify only the correct region associated with the ICAM1 promoter and was modified from. An initial cycle of 95° C. for 3 minutes was followed by a touchdown PCR (95° C. for 1 minute, annealing temperature for 1 minute, 72° C. for 1 minute). The annealing temperature started at 64° C. and was dropped 2° C. degrees after two cycles and then decreased 1° C. after every other cycle until the annealing temperature reached 57° C. After the touchdown PCR, an additional 40 cycles were carried out with the parameters above and the annealing temperature of 56° C.

Amplified PCR products were purified, ligated into pDIM-N plasmids and transformed into NEBS alpha or NEB10 beta cells. Colony PCR identified colonies containing the insert and these colonies were sent for sequencing. The sense strand was amplified with primers 5′-TAG TGA GCG GCC GCT AAG TTG GAG AGG GAG GAT TTG A-3′ (Fw) (SEQ ID NO: 14) and 5′-TAG TTT GAA TTC CAT AAA CAA CTA CCT AAA CAT ACA TAA CCT AACC-3′(Rev) (SEQ ID NO: 15). The anti-sense strand was amplified with primers 5′-TGA GTG CGG CCG CAT AAA ATA AAC ACA ATA ACA ATC TCC ACT CTC-3′(Fw) (SEQ ID NO: 16) and 5′-TTG TAT GAA TTC AGG TTG TAA TTT TGA GTA GTA GAG GAG TTT AG-3′ (Rev) (SEQ ID NO: 17).

Cell lysis and western blot analysis. At 72 hours after transfection, HEK293 cells in 6-well plates were washed in ice cold DPBS and lysed in 50 μl ice cold Ripa lysis buffer (per well) supplemented with 1× protease inhibitor cocktail P8340 (Sigma Aldrich, St. Louis, Mo.). Lysates were vortexed intermittently and incubated on ice for 30 minutes before the soluble fraction was recovered by centrifugation. A 26 μl aliquot of soluble fraction was mixed with 10 μl of 4× NuPage LDS Sample Buffer (Life Technologies, Carlsbad, Calif.) and 4 μl DTT (0.5 M) and incubated at over 70° C. for 10 minutes. Samples were loaded on a 4-12% bis-tris gel and run in MES running buffer supplemented with 500 μl NuPAGE Antioxidant (Life Technologies, Carlsbad, Calif.) at 190 volts for 40 minutes.

Proteins were transferred to PVDF membranes using a Trans-Blot SD Semi-Dry Electrophoretic Transfer Cell (Biorad, Hercules, Calif.) in transfer buffer (10 ml of 20× NuPAGE transfer buffer, 100 μl NuPAGE antioxidant, 10 ml methanol in 100 ml) at 15 V for 30 minutes. The membrane was incubated with anti-flag monoclonal antibody (cat #0420 Lifetein, South Plainfield, NJ) diluted 2000-fold in blocking buffer (5% w/v milk in TBST) overnight at 4° C. The membrane was washed several times in TBST and incubated at room temperature for 30 minutes with a goat anti-mouse-HRP conjugate (cat#170-5047, Biorad, Hercules, Calif.) diluted 6000-fold in blocking buffer (0.4% w/v dry milk in TBST) in a SNAP I.D. system (Millipore, Billerica, Mass.). After washing the membrane in TBST, the membrane was developed using the Immun-Star WesternC Chemiluminescence Kit (Biorad, Hercules, Calif.). Images were taken using the Molecular Imager XRS Gel Doc system and analyzed with Quantity One software.

Construction of Cassette Mutagenesis Library. An NNK cassette mutagenesis library of M.SssI [aa273-386] (SEQ ID NO: 13) was constructed by overlap extension PCR. PCR was carried out using an oligonucleotide degenerate for a five amino acid region in the C-terminal fragment corresponding to amino acids 297-301 in the wild type enzyme. Fragments were digested with AgeI-HF and Spel and ligated into pDIMN8 containing HS2 and the complete N-terminal fragment-HS1 fusion. Site 1 (i.e. the target site in FIG. 1C) contained an FspI site flanked by HS1 and HS2 zinc finger recognition sites. The plasmid also possessed a non-target site that lacked zinc finger binding sites but contained an internal SnaBI restriction site (red site in FIG. 2A). Ligations were transformed into ER2267 electrocompetent cells, which were plated onto agarose plates containing 100 μg/ml ampicillin and 2% w/v glucose. Plates were incubated overnight at 37° C. The naive library contained 2×10⁵ transformants.

Library Selection. Plated library variants were recovered from the plate in lysogeny broth supplemented with 15% v/v glycerol and 2% w/v glucose and stored at −80° C. Aliquots were thawed and used to inoculate 10 ml of lysogeny broth supplemented with 100 μg/ml ampicillin salt, 0.2% w/v glucose, 1 mM IPTG, and 0.0167% w/v arabinose. These cultures were incubated overnight at 37° C. and 250 rpm. Plasmid DNA was isolated via QlAprep Spin Miniprep Kit and digested for 3 hours at 37° C. with McrBC (10 units/μg DNA), FspI (2.5-5 units/μg DNA) in 1× NEBuffer 2 supplemented with 100 μg/ml BSA and 1 mM GTP. Reactions were halted by incubation at 65° C. for over 20 minutes to which ExoIII (30 units/μl DNA) was added and the solution incubated at 37° C. for 60 minutes. ExoIII digestion was halted by incubation at 80° C. for over 30 minutes and the DNA was desalted using Zymo Clean and Concentrator-5 kits per manufacturer's instructions. DNA was transformed into ER2267 electrocompetent cells and plated on agar supplemented with 2% w/v glucose and 100 μg/ml ampicillin salt.

Cells were recovered from the plate as before and plasmid DNA was isolated using the QlAprep Spin Miniprep Kit. The DNA was digested with FspI (2-2.8 units/μg DNA) in 1× NEBuffer 4 and linear DNA was isolated via gel electrophoresis. PCR was used to amplify the portion of the linear plasmid containing genes encoding for the N-terminal and C-terminal fragments fused to zinc fingers. Purified PCR products were subcloned into the selection plasmid for an additional round of selection.

Restriction Endonuclease Protection Assays. Cultures from colonies were incubated overnight at 37° C. and 250 rpm in lysogeny broth supplemented with 0.2% w/v glucose and 100 μg/ml ampicillin salt and stored as glycerol stocks. Glycerol stocks were used to inoculate 10 ml of lysogeny broth supplemented with 100 μg/ml ampicillin salt, 0.2% w/v glucose, 1 mM IPTG, and 0.0167% w/v arabinose. After growth overnight at 37° C., plasmid DNA was purified from the cultures with a QlAprep Spin Miniprep Kit. Plasmid DNA (500 ng) was digested with NcoI-HF (10 units) and either FspI (2.5 units) or SnaBI (2.5 units) in 1× NEBuffer 4 for over one hour at 37° C. SnaBI digests were supplemented with 100 μg/ml BSA. Half of each digested sample was loaded onto agarose gels (1.2% w/v in TAE) and electrophoresed at 90 V for 105-120 minutes. Bands were quantified as described.

Bisulfite Analysis. Glycerol stocks of ER2267 cells containing the methyltransferase variants were used to inoculate 10 ml of lysogeny broth supplemented with 100 μg/ml ampicillin salt, 0.2% w/v glucose, 1 mM IPTG, and 0.0167% w/v arabinose. Cultures were incubated for 12-14 hours at 37° C. and 250 rpm, and the plasmid DNA was isolated. Plasmids (2 μg) were linearized with 1× NcoI-HF (20 Units/ug DNA) in 1× CutSmart Buffer. Linear plasmids were purified using DNA Clean & Concentrator-5 (Zymo Research, Irvine, Calif.). Linearized plasmids (500 ng) were treated with bisulfate reagent using the EZ-DNA Methylation Gold Kit (Zymo Research, Irvine, Calif.). Touchdown PCR, using PfuTurbo Cx Hotstart DNA polymerase was used to amplify regions encoding the target and the non-target sites and was modified from (Immunol. Cell Biol., 79: 18-22. doi:10.1046/j.1440-1711.2001.00968.x.). An initial cycle of 95° C. for 3 minutes was followed by a touchdown PCR (95° C. for 1 minute, annealing temperature for 1 minute, 72° C. for 2 minutes). The annealing temperature started at 64° C. and was dropped 2° C. degrees after two cycles and then decreased 1° C. after every other cycle until the annealing temperature reached 52° C. After the touchdown PCR, an additional 30 cycles were carried out with the parameters above and an annealing temperature of 51° C. A final extension was carried out at 72° C. for 10 minutes. The antisense strand at the target site was amplified with primers 5′-AAG ACA GAG CTC AAA CTA AAT AAC CTT CCC CAT TAT AAT TCT TCT-3′(Fw) (SEQ ID NO: 25) and 5′-CCG TAG CCA TGG TAT ATT TTT AAT AAA TTT TTT AGG GAA ATA GGT TAG GTT TTT AT-3′ (Rev) (SEQ ID NO: 26). The antisense strand at the non-target site was amplified with primers 5′-AAG ACA GAG CTC CTC TAC TAA TCC TAT TAC CAA TAA CTA CTA CCA ATA A-3′(Fw) (SEQ ID NO: 27) and 5′-CCG TAG CCA TGG GTA AAG TTT GGG GTG TTT AAT GAG TGA GTT AAT TTA TAT TAA TTG-3′ (Rev) (SEQ ID NO: 28). PCR amplified products were purified by gel electrophoresis as above digested with SacI-HF and NcoI-HF, ligated into pDIMN9 and transformed into NEB 5-alpha competent E. coli (High Efficiency). Individual colonies were sent for sequencing and analyzed using quantification tool for methylation analysis (QUMA)(Nucleic Acids Res., 36: W170-W175. doi:10.1093/nar/gkn294). Low quality sequences were excluded if they had more than 5 unconverted CpH sites or if less than 95% of all CpH sites were converted. Sequences were also excluded if they either had over 10 alignment mismatches or less than 90% percent identity to the reference sequence.

### Example 1

Library Selections. Initial selection experiments on the library resulted primarily in the isolation of plasmid DNA with a deleted FspI restriction site, presumably formed by a recombination event. This false positive was a trivial, albeit frequently observed, solution for plasmid survival in the inventive system. Thus, the plasmid DNA from the resulting transformants was subjected to additional steps to enrich for those plasmids that survived the selection and retained their FspI site. In these additional steps, the plasmid DNA was transformed into ER2267 cells and the cells were plated under conditions known to repress the promoters controlling methyltransferase fragment expression. Plasmid DNA from these cells was digested with FspI and the linear, FspI-digested DNA was purified away from undigested plasmid DNA by agarose gel electrophoresis. The portion of the plasmid encoding the zinc fingers and methyltransferase genes was PCR amplified, ligated back into the same plasmid backbone, and subjected to an additional round of selection. The additional round of selection also included this FspI site-enrichment step. Variants were then selected for further analysis.

### Example 2

Analysis of Library Variants that Survived the Selection. 47 variants were assayed for methylation activity at both the target and non-target site and determined the variants' sequences. For some constructs, the non-target site's SnaBI restriction site was replaced with an FspI site, allowing the quantification of the target and non-target methylated bands more easily (not shown). The variants (e.g. having the amino acid sequences PFCSY (SEQ ID NO: 18), CFESY (SEQ ID NO: 19), and SYSSS (SEQ ID NO: 20), which are named for the sequence at residues (297-301) of M.SssI methylated 70-80% of the plasmids at the target site with minimal methylation (0-8%) at the non-target site. Representative variants are shown in FIG. 2D. Most active variants displayed biased methyltransferase activity toward the targeted site.

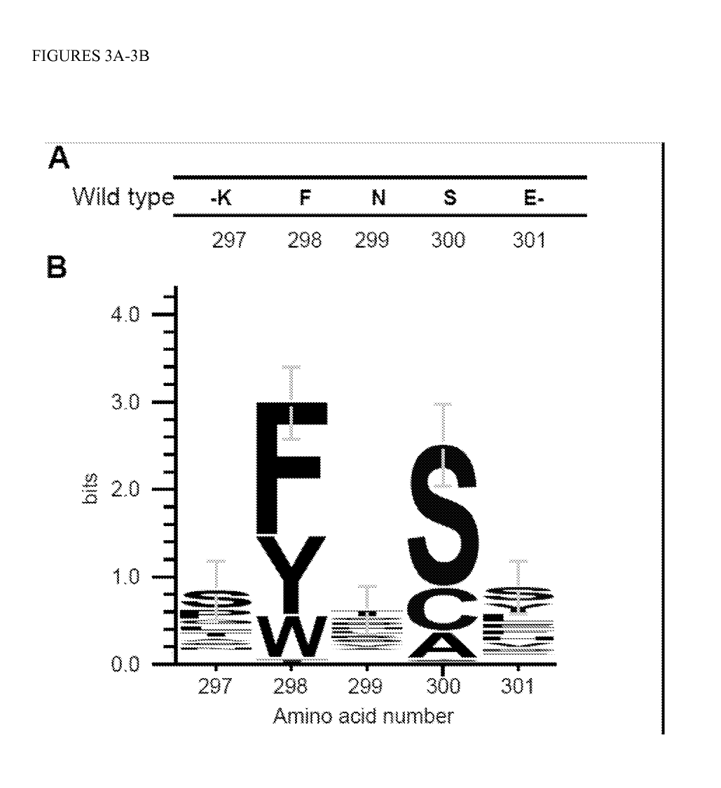

A comparison of the sequences of active variants, using weblogo 3.3, indicated that a functional heterodimeric methyltransferase strongly preferred certain residues at positions 298 and 300 (FIG. 3). Position 298 (wild-type phenylalanine) was almost exclusively composed of aromatic residues. Position 300 (wild-type serine) was almost exclusively composed of small residues. The observed conservation at these residues is consistent with sequence alignments showing these two residues are relatively well-conserved among methyltransferases of different species. In contrast, positions 297, 299 and 301 exhibited little preference for specific amino acids. This finding is consistent with the mutational studies discussed above. The present findings reveal that there are numerous solutions for improving the specificity of the zinc finger-fused, bifurcated methyltransferase fusion proteins of the present invention.

### Example 3

To further characterize some of these fusion protein variants, library fragments were cloned into plasmids containing a control non-target site (lacking both zinc finger binding sites) and a half-site (lacking one of the zinc finger sites) adjacent to the FspI restriction site. As with our previously described split M.HhaI constructs, these split M.SssI constructs did not require the presence of both zinc finger binding sites for methylation activity (data not shown). However, the CFESY and SYSSS constructs exhibited a synergistic activity caused when both zinc finger recognition sites flanked the targeted CpG site. In other words, the observed activity at the full site was greater than the additive effects of each individual half site.

### Example 4

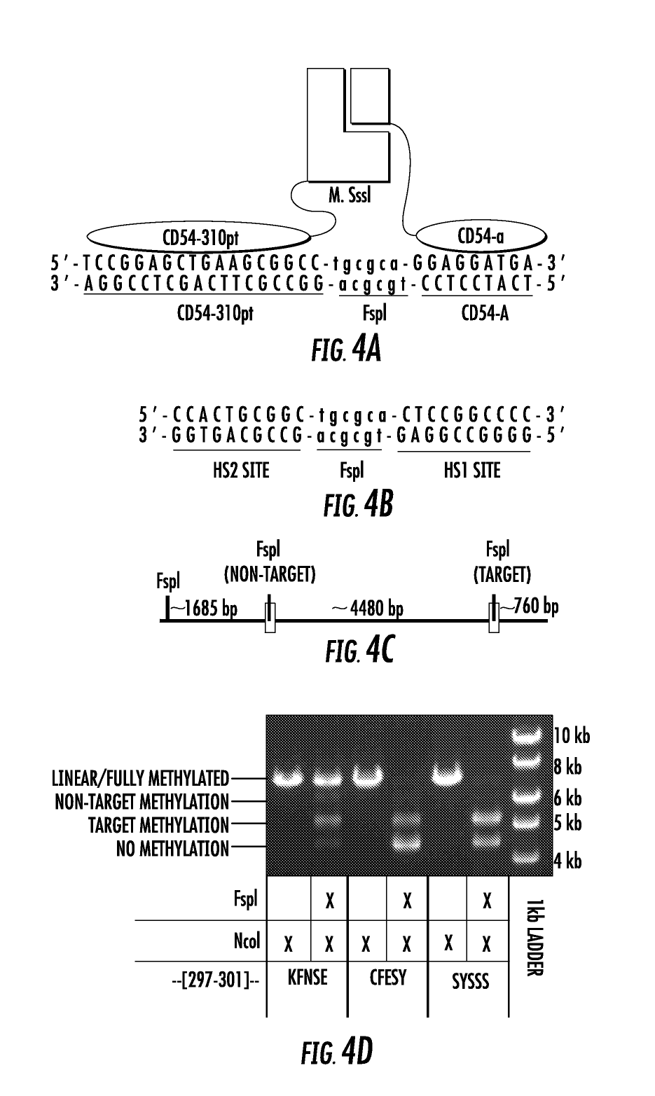

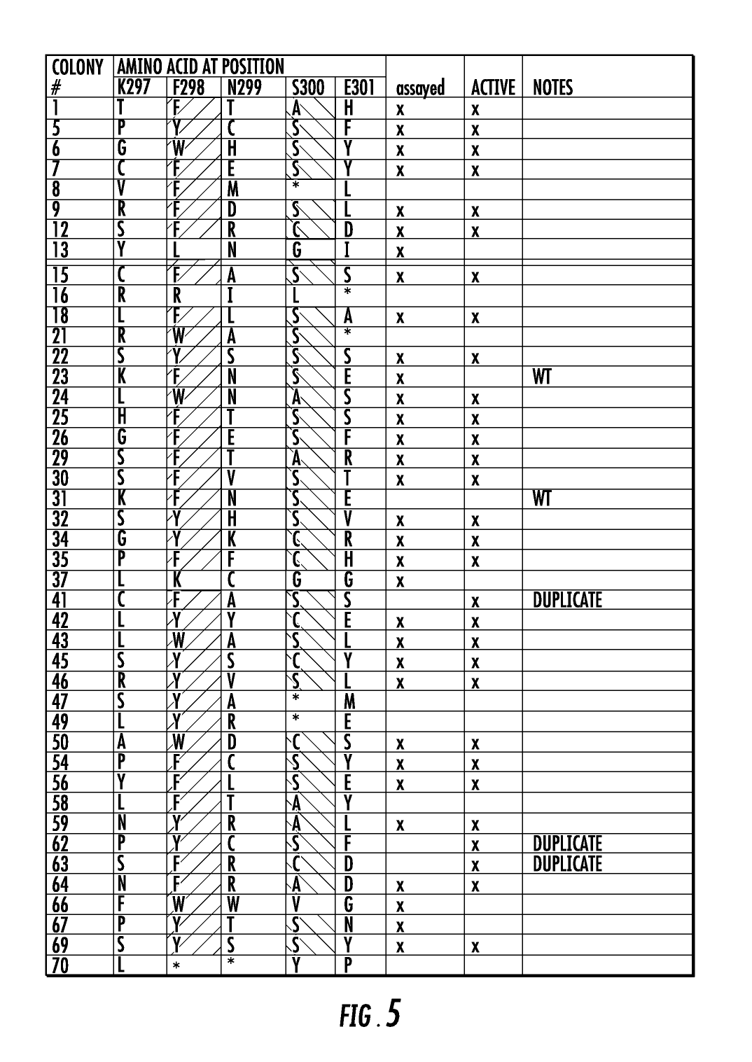

The targeted heterodimeric methyltransferase fusion proteins of the present invention are modular. To test whether or not the targeted M.SssI methyltransferase fusion proteins of the present invention are modular with respect to the zinc finger domains, zinc fingers HS1 (SEQ ID NO: 21) and HS2 (SEQ ID NO: 22) were replaced with two zinc fingers designed to target a specific site in the promoter of intercellular adhesion molecule 1 (ICAM1). The previously designed zinc finger CD54-31Opt (J. Mol. Biol., 341: 635-649 (2004)) (SEQ ID NO: 23) is adjacent to a CpG site in this promoter. To generate a pair of zinc fingers capable of flanking this CpG site, a second zinc finger, CD54a (SEQ ID NO: 24) was designed, to bind downstream from the recognition sequence of CD54-31Opt and adjacent CpG site (FIG. 4A). The two zinc fingers were fused to fragments comprising non-optimized bifurcated M.SssI fragments (residues KFNSE (SEQ ID NO: 7) at positions 297-301) and to two selected variants (CFESY (SEQ ID NO: 19) and SYSSS (SEQ ID NO: 20) at positions 297-301), replacing the HS1 and HS2 zinc fingers (FIG. 4A). These two optimized variants were chosen because methylation at the target site (containing both zinc finger binding sites) was greater than the additive amount of methylation levels observed at half sites, as discussed above.

The sequences of the wild-type zinc finger-fusion protein variants of the present invention are shown in FIG. 5. The methyltransferase activity and specificity of these fusion protein constructs was assessed in E. coli using a restriction endonuclease protection assay (FIGS. 4C, D). Although all three constructs biased methylation to the target site from the ICAM1 promoter, the CFESY and SYSSS constructs targeted methylation to the desired site with little to no observable methylation at the non-target site. Notably, the ‘non-target’ site in this experiment contained the zinc finger sequences recognized by HS 1 and HS2 (FIG. 4B).

The CD54-3 lOpt was chosen because it was shown to effectively target the ICAM1 promoter, altering transcription levels when fused to transcriptional activators or repressors. Additionally, fusion of CD54-3 lOpt to Ten-Eleven Translocation 2 enzyme resulted in a small, observable amount of demethylation around the target site, correlating with a 2-fold upregulation in ICAM1 transcription. Thus, the fusion protein constructs of the present invention can potentially enable assessment of the biological effects of targeted methylation at this and other sites, using the methods described herein.

### Example 5

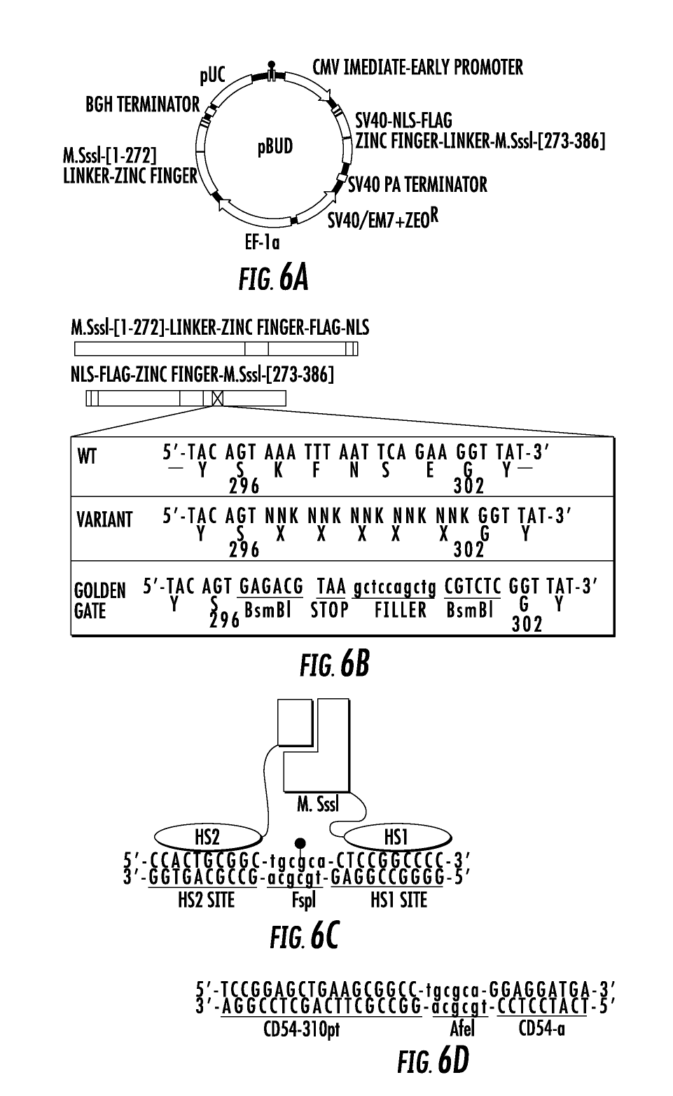

Heterodimeric methyltransferase-fusion proteins target methylation toward specific sites and are expressed in HEK293 cells. We first attempted to demonstrate that methyltransferase fragments can be expressed and can target methylation in HEK293 cells. Each zinc finger methyltransferase fusion construct was cloned under the control of a separate constitutive promoter (FIG. 6A). In these experiments, HS1 and HS2 zinc fingers were fused to N-terminal and C-terminal M.SssI fragments as described herein. Additionally, sequences encoding the SV40 NLS and FLAG tag were fused to the terminal ends of each zinc finger (FIG. 6B). Finally, we added a targeted CpG site, nested within an FspI restriction site, flanked by HS1 and HS2 recognition sequences (FIG. 6C). Transient transfection of pBUD plasmid containing an unrelated gene, Haps59-EGFP fusion (Proc. Natl. Acad. Sci., USA 108: 16206-16211 (2011)), demonstrated that under the conditions used to transfect our methyltransferase variants, 75-80% of the Haps59-EGFP transfected cells were fluorescent 72 hours post-transfection.

The plasmids expressing methyltransferase fragments were isolated 72 hours after transfection. Transfected plasmids and non-transfected plasmids were assayed for their sensitivity to endonucleases whose activity is blocked by CpG methylation. Similar to the E. coli expression described above, the targeted CpG site is nested within an FspI site. A SnaBI restriction site, present in the CMV-promoter is not flanked by these zinc finger binding recognition sequences and is considered a non-target site. Thus, nicked or supercoiled plasmid in FspI or SnaBI digestion lanes indicates methylation-dependent protection at the target or non-target sites, respectively.

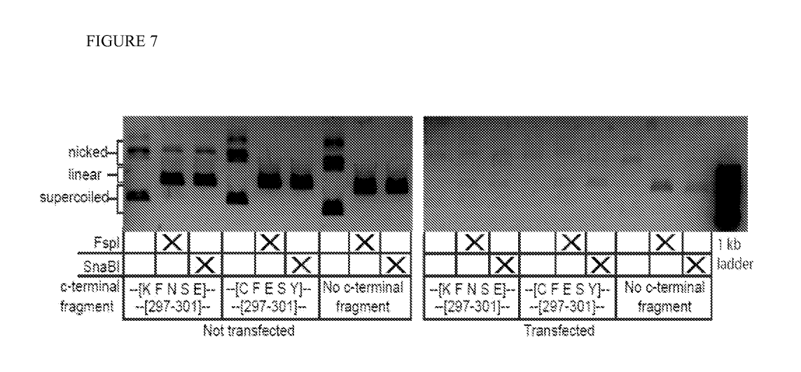

Results demonstrated that the plasmid DNA, prior to transfection, was sensitive to SnaBI and FspI digestion. This is expected because the pBUD plasmid lacks promoters recognized by native E. coli transcription machinery; methyltransferase fragments, therefore, should not be actively expressed in the E. coli from which the plasmid DNA was prepared. However, plasmid DNA encoding ‘wild-type’ (i.e. no mutations to residues 297-301) methyltransferase fragments appear to be partially protected from digestion prior to transfection (as indicated by nicked DNA in FIG. 7, panel 1). This may be due to low-level, leaky transcription of these highly active methyltransferase fragments in E. coli. Regardless, the ratio of protected DNA to digested DNA was so low that this was not expected to alter the interpretation of the protection assays in transfected plasmids. Undigested, non-transfected plasmids were present in nicked and supercoiled forms. In this case, the high levels of nicked DNA may result from the isolation procedure or from the use of zeocin, a DNA damaging agent, as a selectable marker during preparation in E. coli.

For plasmid isolated from transfected cells, the ‘wild-type’ heterodimeric methyltransferase fusion protein (KFNSE (SEQ ID NO: 7) in the region corresponding to aa 297-301) methylates equally at the target and non-target site, as indicated by the increased presence of nicked DNA relative to linear DNA (FIG. 7, panel 2). The lack of specificity for the target site over non-target site in HEK293 cells mirrors the lack of specificity observed in E. coli. Similar to our in vivo E. coli experiments, in HEK293 cells the optimized variant, with residues CFESY (SEQ ID NO: 19) in the region corresponding to aa 297-301, appears only methylated at the target site. This result is indicated by the presence of nicked band in the FspI digested, but not the SnaBI digested lanes (FIG. 7 panel 2). As expected, plasmid lacking one of the two obligate heterodimeric fragments shows no nicked or supercoiled DNA when digested with either FspI or SnaBI. However, unlike the results in E. coli, we observed large amount of unprotected plasmid DNA in our transfected ‘wild-type’ constructs. This may be due to inefficient transcription or translation of the methyltransferase fragments in our transfected cells. Further, incomplete methylation may also be due to a limited number of plasmids present in the nucleus compared to the cytoplasm.

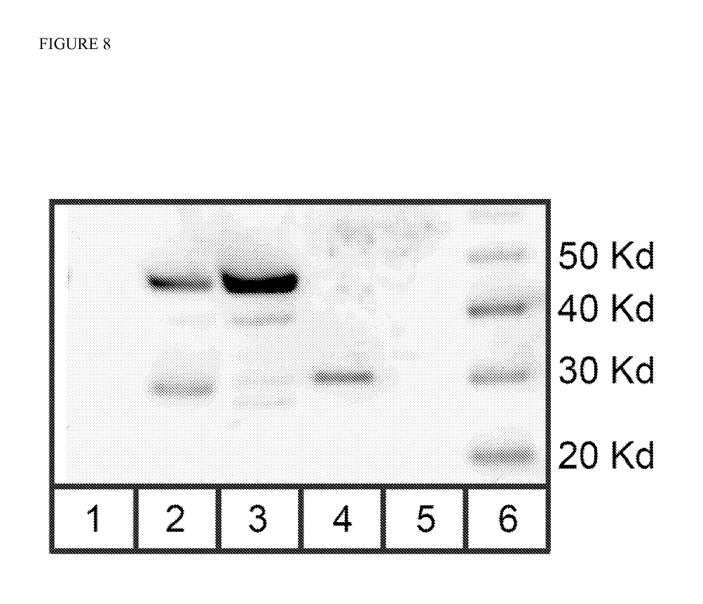

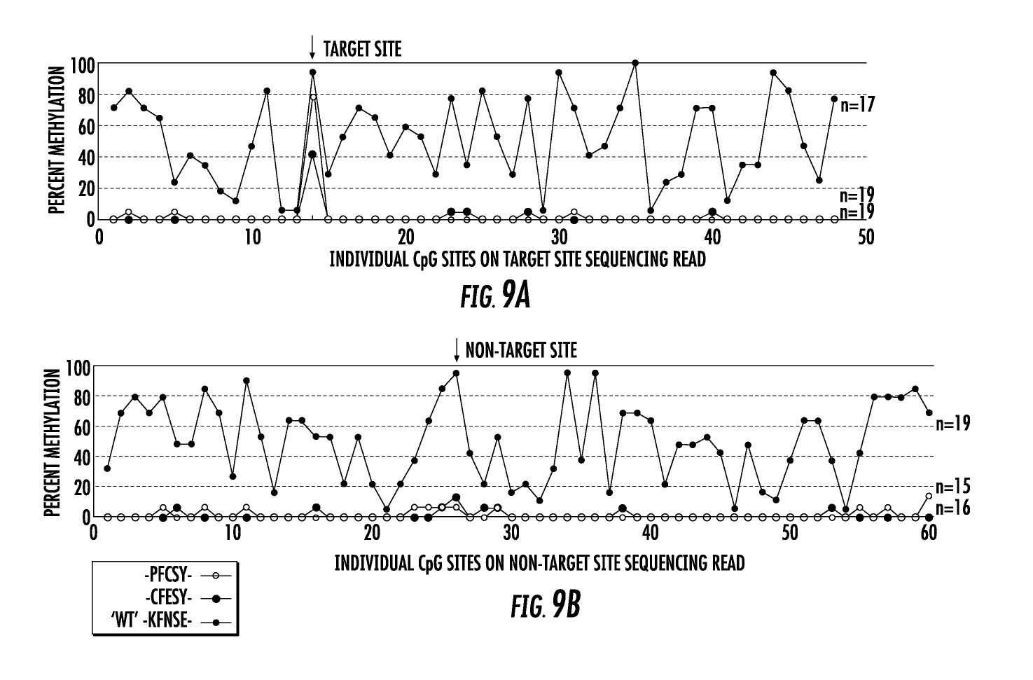

To further demonstrate that both fragments were expressed in at least some population of HEK293 cells, transiently transfected cells were lysed 72 hours after transfection. A western blot of the lysates using anti-FLAG-tag antibodies revealed that cells transfected with the ‘wild-type’ N-terminal and C-terminal methyltransferase-zinc finger fusion protein fragments produced two bands of the expected sizes (45 Kd and 25.8 Kd respectively) (FIG. 8). Cells transfected with plasmid encoding only the N-terminal fragment expressed only one band (45 Kd) of the expected size.

### Example 6

‘Wild-type’ heterodimeric zinc finger fusion proteinsof the present invention methylate chromosomal DNA. It would be significant to show that a heterodimeric methyltransferase is active on chromosomal DNA. Studies have shown that zinc fingers known to interact with plasmid DNA may not be able to access the same sequences within the chromosome due to the DNA's inaccessibility within the chromatin structure.

To demonstrate that the heterodimeric-zinc finger methyltransferases are active on the on the chromosome, pBUD plasmids containing zinc finger methyltransferase fusion proteins were transfected into RKO cells. In these experiments, the N-terminal construct was fused to CD54-31 Opt and the C-terminal constructs were fused to CD54a (as described above). A target site with cognate zinc finger binding sequences flanking an internal AfeI site was also cloned into these vectors (FIG. 6D). These constructs were used because they encode zinc fingers that, in E. coli, efficiently targeted methylation to a region of DNA matching one found in the promoter of the Intercellular Cell Adhesion Molecule 1 (ICAM1) gene. Further, the promoter of ICAM1 was found to be hypomethylated in RKO cells. Preliminary bisulfite analysis confirmed this.

Bisulfite sequencing of the antisense strand (relative to the top strand in FIG. 7D) reliably covers 29 CpG sites. When we analyzed 8 clones from bisulfite treated CFESY optimized variant, we observed one methylated site present on one of the 8 clones. This site was not the CpG site flanked by the zinc finger recognition sequences. When we assessed chromosome isolated from cells transected with the ‘wild-type’ variant, 4 of 15 clones had methylation at least two sites. One clone was methylated at 16 of the possible 29 sites assessed. Only one sequence appeared methylated at the target site.

The results are the first evidence to suggest that the heterodimeric methyltransferase fusion proteins of the present invention can methylate chromosomal DNA. The transfection efficiency was estimated qualitatively to be 30-40% based on fluorescence of a pBUD Haps59-EGFP construct that was transfected under the same conditions. Assuming the transfection efficiency of the active ‘wild-type’ methyltransferases is the same, than all successfully transfected cells showed some degree of methylation.

### Example 7

To further characterize the engineered methyltransferases of the present invention, plasmids containing optimized variants, PFCSY, CFESY (named for the sequence at residues 297-301), and the un-optimized ‘WT’ variant, were subjected to bisulfite analysis at both the target and non-target sites. These plasmids were isolated from cultures in which the methyltransferase fragments were expressed. The region subjected to bisulfite sequencing includes 47 and 59 CpG sites around the target and non-target sites, respectively (covering over 25% of the total CpG sites present on the plasmid) in addition to the target and non-target CpG sites. At least 15 or more clones for each variant were sequenced to quantify the frequency of methylation at all CpG sequences around both sites (FIGS. 9A, B). Based on this sequencing, the PFCSY variant methylated the target site at a frequency of 78.9%. In contrast, only fifteen off-target methylation events were observed in the 34 sequence reads (out of a total of 1793 possible off-target methylation events), which corresponds to an off-target methylation frequency of 0.84%. This specificity for the target site is a significant improvement over the un-optimized, ‘WT’ variant, which methylated the target site at a frequency of 94.1% and off-target sites at a frequency of 49.5%. Thus, for this variant, the selections resulted in the identification of a variant with an almost 60-fold reduction in off-target methylation yet a minimal decrease in methylation at the target site. The CFESY variant was somewhat less capable of methylating the target site compared to the PFCSY variant, but exhibited a similar low frequency of methylation at other CpG sites (target frequency of 42.1% and a 0.71% frequency at all other CpG sites).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A fusion protein comprising:
a) a polypeptide encoding an N-terminal portion of M.SssI methyltransferase;
b) a polypeptide encoding a first DNA binding peptide specific for a DNA sequence of interest;
c) a peptide encoding a first linker molecule which is covalently linked to the N-terminal portion of M.SssI methyltransferase and the first DNA binding peptide;
d) a polypeptide encoding a C-terminal portion of M.SssI methyltransferase, wherein the C-terminal portion encodes a mutation;
e) a polypeptide encoding a second DNA binding peptide specific for a DNA sequence of interest; and
f) a peptide encoding a second linker molecule which is covalently linked to the C-terminal portion of the M.SssI methyltransferase and the second DNA binding peptide.

2. The fusion protein of claim 1, wherein when the fusion protein is expressed, the fusion protein is capable of methylation of a target CpG site.

3. The fusion protein of claim 2, wherein the polypeptide of a) comprises amino acid residues 1-272 of M.SssI methyltransferase.

4. The fusion protein of claim 3, wherein the polypeptide of d) comprises amino acid residues 237-386 of M.SssI methyltransferase having a mutation of up to five amino acids at residues 297-301.

5. (canceled)

6. The fusion protein of claim 3, wherein the first and second DNA binding peptides are polypeptides which encode a zinc finger domain.

7. The fusion protein of claim 1, comprising the amino acid sequence of SEQ ID NOS: 1 or 2.

8. The fusion protein of claim 6, wherein the DNA binding polypeptides comprise zinc finger binding domains selected from the group consisting of HS 1, HS2, CD54-31Opt, and CD54a.

9. The fusion protein of claim 8, wherein the five mutated amino acids are residues 297-301 of the M.SssI methyltransferase, and have the sequence AA₁-AA₂-AA₃-AA₄-AA₅, wherein each of the AAₙ can be any amino acid, with the proviso that the amino acid sequence cannot be K-F-N-S-E.

10. The fusion protein of claim 9, wherein AA₂ is an amino acid residue selected from the group consisting of F, Y and W, and AA₄ is an amino acid residue selected from the group consisting of S, C and A.

11. A nucleic acid molecule encoding the fusion protein of to claim 1.

12. The nucleic acid molecule of claim 11, comprising the nucleic acid sequence of SEQ ID NOS: 3 or 4.

13. An expression vector comprising the nucleic acid molecule of claim 12.

14. The expression vector of claim 13, comprising the nucleic acid sequence of SEQ ID NOS: 5 or 6.

15. A micro-organism transformed with the expression vector of claim 14.

16. A method for selection of a fusion protein comprising a methyltransferase having specificity for a methylation site of interest, comprising:
an E. coli cell transformed with the expression vector of either of claim 13, wherein the expression vector comprises a restriction enzyme site having a target methylation site within the nucleic acid sequence of the restriction enzyme site, and wherein the restriction enzyme specific for said site can only cleave the restriction site in the absence of CpG methylation, and wherein the vector encodes DNA sequences which flank the restriction site that are specific for the DNA binding peptides encoded in the vector;
expressing the polypeptides encoded by the vector in the E. coli cell;
allowing the vector to become methylated by the methytransferase encoded by the vector;
isolating the DNA of the vector;
digesting the DNA of the vector in vitro with an endonuclease specific for said restriction site and with the endonuclease McrBC;
incubating the vector DNA with the enzyme ExoIII; and
isolating and purifying the remaining intact vectors.

17. The method of claim 16 wherein the endonuclease is FspI and the restriction site in the vector is specifically cleaved by FspI.

18. The method of claim 17, wherein the DNA binding polypeptides in the vector are selected from the group consisting of HSP1 and HSP2, and the DNA sequences which flank the restriction site in the vector are specifically bound by HSP1 and HSP2.

19. The method of claim 17, wherein the DNA binding polypeptides in the vector are selected from the group consisting of CD54-31Opt and CD54a, and the DNA sequences which flank the restriction site in the vector are specifically bound by CD54-31Opt and CD54a.
